(19) United States
(12) Patent Application Publication
(10) Pub. No.: US 2020/0085857 A1
(43) Pub. Date: Mar. 19, 2020

(54) COMPOSITION FOR ALLEVIATING ATOPIC SKIN SYMPTOMS, CONTAINING POLY-GAMMA-GLUTAMIC ACID

(71) Applicant: Bioleaders Corporation, Gyeonggi-Do (KR)

(72) Inventors: Jae-Chul Choi, Daejeon (KR); IL-Han Lee, Yongin-si (KR); Heejin Cho, Hanam-si (KR); Seung Hoon Kim, Seoul (KR)

(73) Assignee: Bioleaders Corporation, Gyeonggi-Do (KR)

(21) Appl. No.: 16/470,890

(22) PCT Filed: Dec. 15, 2017
(86) PCT No.: PCT/KR2017/014880

§ 371 Date: Jun. 18, 2019

(30) Foreign Application Priority Data
Dec. 19, 2016 (KR) 10-2016-0173380

(51) Int. Cl. A61K 31/785; A61P 17/00; A61P 29/00; A61K 9/00

(52) U.S. Cl. CPC A61K 31/785; A61K 9/0014; A61P 29/00; A61P 17/00

(57) ABSTRACT

Disclosed is a composition for alleviating symptoms of atopic dermatitis that contains a high molecular weight poly-gamma-glutamic acid and a low molecular weight poly-gamma-glutamic acid as active components. The composition for improving symptoms of atopic dermatitis inhibits epidermal and transepidermal water loss, improves desquamation (scaling skin), provides an excellent skin soothing effect, and relieves pruritus (itch) caused by dryness, thereby making remarkable improvement of general symptoms of atopic dermatitis.

### TECHNICAL FIELD

The present invention relates to a composition containing poly-gamma-glutamic acids for alleviating symptoms of atopic dermatitis, and more particularly to a composition containing a high molecular weight poly-gamma-glutamic acid and a low molecular weight poly-gamma-glutamic acid as active components to provide effective ways to improve the symptoms of atopic dermatitis.

### BACKGROUND ART

Atopic dermatitis is a skin disease that commonly occurs in 0.5 to 1% of the world's population. It affects 5 to 10% of children, typically occurring between 2 and 6 months of age. Most cases of atopic dermatitis develop in the first year of life, and 85% develops by 5 years of age. 50% of those who develop the atopic condition in childhood go into remission in the first two years of life. 25% of the children with atopic dermatitis suffer from this condition also in adolescence and the rest 25% continue to have symptoms into adulthood.

It is not clear what exactly causes atopic dermatitis, although the development of the disease results from genetic hereditary and immune dysfunction. The other known factors that cause atopic dermatitis may include dry skin, an increased tendency toward itching, infections caused by bacteria, viruses, fungi, etc., and a combination of emotional and environmental factors. The most common symptoms are severe pruritus (itch), dry skin, rash, oozing, crusting, and scaling skin. There is currently no cure for atopic dermatitis in the aspect of the modern medical science, so treatments involve avoiding the triggers of atopic dermatitis and helping the patients manage the symptoms, rather than aiming to cure the disease.

The medication-based treatment for atopic dermatitis involves steroid agents for treating itching and dermatitis, non-steroid agents such as antihistamines, antibiotics, and moisturizers. The steroids are adrenocortical hormones that have anti-inflammatory and immunosuppressive functions. They are excellent in the effects, but their prolonged use may result in adverse effects, which include excessive hair growth on the skin being treated, skin atrophy, hypopigmentation, skin infection, acne, thinning of the skin to make the vein seen clearly. The antihistamines are used as a temporary measure to relieve itching by blocking the release of histamines from mast cells. A prolonged use of antihistamine may cause side effects, including sleep difficulty, restlessness, dizziness, loss of appetite, and so forth. The antibiotics are used to treat a secondary infection caused by bacteria like Staphylococci that occurs in scratched areas of itchy skin, so they are not considered as a fundamental cure for atopic dermatitis.

Poly-gamma-glutamic acid is a mucilaginous polymer of amino acids that comprises a gamma linkage of glutamic acids and is produced by Bacillus sp. The inventors of the present invention have patented a high molecular weight poly-gamma-glutamic acid and a method of using the same (Korean Patent Registration No. 399091); a method of producing a poly-gamma-glutamic acid using anti-inflammatory Bacillus subtilis strain derived from Cheongguk-jang (fast-fermented bean paste) to produce high molecular weight poly-gamma-glutamic acids (Korean Patent Registration No. 500796); and an anti-carcinogenic composition, an immunoadjuvant and an immune booster that contain a poly-gamma-glutamic acid (Korean Patent Registration Nos. 496606, 517114 and 475406). In addition, the inventors of the present invention have unveiled the anti-carcinogenic functions of hyaluronidase inhibitors containing a poly-gamma-glutamic acid (Korean Patent Registration No. 582120) and poly-gamma-glutamic acids (Poo, H. R. et al., Journal of Immunology, 178:775, 2007) through reinforcing immunity, which boosted the studies on the medicinal use of the poly-gamma-glutamic acids. Hence, the different efficacies of poly-gamma-glutamic acids have been discovered through a sustained development of the use of poly-gamma-glutamic acids.

In an effort to develop a composition for effectively improving the symptoms of atopic dermatitis, the inventors of the present invention have found it out that the application of a composition containing both a high molecular weight poly-gamma-glutamic acid and a low molecular weight poly-gamma-glutamic acid to a patient with atopic dermatitis can prevent a loss of water in the skin, reduce desquamation (scaling skin), provide a skin soothing effect, and relieve pruritus (itch) caused by dryness, thereby completing the present invention.

### DISCLOSURE

### Technical Problem

It is an object of the present invention to provide a composition for effectively improving the symptoms of atopic dermatitis.

### Technical Solution

To achieve the object of the present invention, there is provided a composition for topical use for improving the symptoms of atopic dermatitis that contains a high molecular weight poly-gamma-glutamic acid and a low molecular weight poly-gamma-glutamic acid as active components.

In another aspect of the present invention, there is also provided a method for treating or improving the symptoms of atopic dermatitis that includes applying a composition for topical use for improving the symptoms of atopic dermatitis that contains a high molecular weight poly-gamma-glutamic acid and a low molecular weight poly-gamma-glutamic acid as active components.

### PREFERRED EMBODIMENTS OF THE PRESENT INVENTION

The present invention determines the molecular weight and the content ratio of poly-gamma-glutamic acids that optimally promote the production of skin-moisturizing factors, such as hyaluronic acid, urocanic acid, lactic acid, etc., and proteins having a skin barrier function, such as filaggrin, involucrin, loricrin, and transglutaminase.

In addition, the present invention provides an assessment of the composition containing both a high molecular weight poly-gamma-glutamic acid and a low molecular weight poly-gamma-glutamic acid in regards to the effects on the human body to improve epidermal water content, transepidermal water loss and desquamation (scaling skin), provide a skin soothing effect, and relieve pruritus (itch) caused by dryness.

Accordingly, the present invention is directed to a composition for topical use for improving the symptoms of atopic dermatitis that contains a high molecular weight poly-gamma-glutamic acid and a low molecular weight poly-gamma-glutamic acid as active components.

In the present invention, the high molecular weight poly-gamma-glutamic acid and the low molecular weight poly-gamma-glutamic acid are preferably contained at a ratio of 1:1 to 1:5.

The composition has an insignificant therapeutic effect when the ratio of the high molecular weight poly-gamma-glutamic acid to the low molecular weight poly-gamma-glutamic acid is less than 1:1; and the composition makes an insignificant moisturizing effect, but becomes hard to be made into a formulation when the ratio of the high molecular weight poly-gamma-glutamic acid to the low molecular weight poly-gamma-glutamic acid is greater than 1:5.

In one aspect of the present invention, the molecular weight of the high molecular weight poly-gamma-glutamic acid is preferably 1,500 to 3,500 kDa, more preferably 2,000 to 2,500 kDa.

In another aspect of the present invention, the molecular weight of the low molecular weight poly-gamma-glutamic acid is preferably 0.5 to 2 kDa, more preferably 1 to 1.5 kDa.

Only when containing the high molecular weight poly-gamma-glutamic acid and the low molecular weight poly-gamma-glutamic acid in the desirable molecular weight range, the composition for topical use according to the present invention can optimally promote the production of skin-moisturizing factors, such as hyaluronic acid, urocanic acid, lactic acid, etc., and proteins having a skin barrier function, such as filaggrin, involucrin, loricrin, and transglutaminase and thereby make a great effect to improve the symptoms of atopic dermatitis.

In the present invention, the high molecular weight poly-gamma-glutamic acid is contained in an amount of 0.01 to 0.5 wt. % with respect to the total weight of the composition; and the low molecular weight poly-gamma-glutamic acid is contained in an amount of 0.01 to 2.5 wt. % with respect to the total weight of the composition.

The content of the high molecular weight poly-gamma-glutamic acid less than 0.01 wt. % results in having the composition make an insignificant moisturizing effect; and the content of the high molecular weight poly-gamma-glutamic acid greater than 0.5 wt. % reduces the viscosity and makes the composition difficult to be made into formulation.

The content of the low molecular weight poly-gamma-glutamic acid less than 0.01 wt. % makes the therapeutic effect of the composition insignificant; and the content of the low molecular weight poly-gamma-glutamic acid greater than 2.5 wt. % causes skin irritations such as itching or tingling of skin.

The composition for topical use according to the present invention may include at least one non-toxic, pharmaceutically acceptable carrier, adjuvant, diluent, or other active ingredients generally available in the related art. The composition for topical use according to the present invention may be formulated by a known method using pharmaceutically acceptable carriers and excipients.

For an example, the composition for topical use according to the present invention may be prepared into any appropriate formulation type as a pharmaceutical preparation, including topical formulations, such as ointment, gel, cream, patch, spray, etc. The individual formulations may contain any kind of substrates and additives necessary and appropriate to the preparation of each formulation type, and the inventors of the present invention may choose the type and content of these components with ease.

When the composition for topical use according to the present invention is prepared into a cosmetic formulation, the formulation type available is not specifically limited and may include skin softener, toner, nutrition lotion, eye cream, nutrition cream, massage cream, cleansing cream, foaming cleanser, cleansing water, powder, essence, facial pack, and so forth.

The composition of the present invention helps improve epidermal water content, transepidermal water loss and desquamation (scaling skin), provide a skin soothing effect and relieve pruritus (itch) caused by dryness and makes a great effect to improve the symptoms of atopic dermatitis, which has been proved by the fact that it causes no skin irritation in patients with atopic dermatitis during the assessment tests.

Hereinafter, the disclosure of the present invention will be described in further detail with reference to examples, which are given for the understanding of the disclosure of the present invention and not intended to limit the scope of the claims in the present invention.

### Examples 1, 2 and 3, and Comparative Examples 1 and 2

A high molecular weight poly-gamma-glutamic acid (2,000 kDa; hereinafter, referred to as “HM-PGA”) and a low molecular weight poly-gamma-glutamic acid (1 kDa; hereinafter, referred to as “LM-PGA”) were added to the base material of the existing Doctors-PGA cream (BioLeaders Corp., South Korea) excluding poly-gamma-glutamic acids at a content ratio specified in the following Table 1 to prepare cream type preparations according to Examples 1, 2 and 3, and Comparative Examples 1 and 2.

TABLE 1

| | | | Content ratio of |
|---|---|---|---|
| Div. | HW-PGA | LW-PGA | HW-PGA TO LW-PGA |
| Example 1 | 0.3 wt. % | 0.3 wt. % | 1:1 |
| Example 2 | 0.3 wt. % | 0.9 wt. % | 1:3 |
| Example 3 | 0.3 wt. % | 1.5 wt. % | 1:5 |
| Comparative | 0.3 wt. % | — | HW-PGA alone |
| Example 1 | | | |
| Comparative | — | 0.3 wt. % | LW-PGA alone |
| Example 2 | | | |

### Experimental Example 1

Assessment of Formulations Containing HM-PGA or LM-PGA Alone or in Combination in Regards to Effect of Improving Symptoms of Atopic Dermatitis

The individual formulations of Examples 1, 2 and 3 and Comparative Examples 1 and 2 were applied to 10 participants (male or female, differently aged) with the symptoms of atopic dermatitis over a 7-day period. Each participant was asked to perform a self-assessment for the intensity and frequency of itch and the intensity of itch during sleep before and after using the formulations. The assessment results are presented in the following Table 2 (unit: person).

Test Subjects

(1) Sex: males (50%), females (50%)

(2) Age: 5 to 9 years old (50%), 10 to 15 years old (33%), 15 to 19 years old (17%)

TABLE 2
(1) Intensity of itch

| | | Intensity of itch | | | | | | |
|---|---|---|---|---|---|---|---|---|
| Item | | 0 | 1 | 2 | 3 | 4 | 5 | Average |
| Comparative | Before | | | 6 | 3 | 1 | | 2.5 |
| Example 1 | After | | | 6 | 4 | | | 2.4 |
| Comparative | Before | | | 7 | 1 | 2 | | 2.5 |
| Example 2 | After | | | 7 | 2 | 1 | | 2.4 |
| Example 1 | Before | | | 5 | 4 | 1 | | 2.6 |
| | After | 1 | 6 | 2 | 1 | | | 1.3 |
| Example 2 | Before | | | 7 | 3 | | | 2.3 |
| | After | 2 | 6 | 2 | | | | 1.0 |
| Example 3 | Before | | | 7 | 1 | 2 | | 2.5 |
| | After | 2 | 6 | 1 | 1 | | | 1.1 |

0 = n/a;
1 = nearly feels neither itchy nor unpleasant;
2 = sometimes feels slightly itchy;
3 = frequently feels itchy but pleasant for activity or rest;
4 = frequently feels extreme itching as much as to disturb relaxation but
allow activity; and
5 = always feels extreme itching as much as to disturb both activity and
relaxation.

(2) Frequency of itch

| | | Frequency of itch | | | | | | |
|---|---|---|---|---|---|---|---|---|
| Item | | 0 | 1 | 2 | 3 | 4 | 5 | Average |
| Comparative | Before | | | 6 | 3 | | 1 | 2.6 |
| Example 1 | After | | | 5 | 4 | 1 | | 2.6 |
| Comparative | Before | | | 9 | | 1 | | 2.2 |
| Example 2 | After | | | 8 | 1 | 1 | | 2.3 |
| Example 1 | Before | | | 6 | 2 | 2 | | 2.6 |
| | After | 1 | 7 | 2 | | | | 1.1 |
| Example 2 | Before | | | 8 | 1 | 1 | | 2.3 |
| | After | 2 | 7 | | 1 | | | 1.0 |
| Example 3 | Before | | | 7 | 3 | | | 2.3 |
| | After | 1 | 8 | 1 | | | | 1.0 |

0 = n/a;
1 = 3 or less times a day, lasting for 10 minutes or less on each time;
2 = 4 or more times a day, lasting for 10 minutes or less on each time;
3 = 4 or more times a day, lasting for 15 minutes or longer on each time;
4 = 4 or less times a day, lasting for 30 minutes or less on each time; and
5 = incessant itch

(3) Intensity of itch during sleep

| | | Intensity of itch during sleep | | | | | |
|---|---|---|---|---|---|---|---|
| Item | | 0 | 1 | 2 | 3 | 4 | Average |
| Comparative | Before | | 2 | 5 | 3 | | 2.1 |
| Example 1 | After | | 2 | 6 | 2 | | 2.0 |
| Comparative | Before | | 1 | 7 | 2 | | 2.1 |
| Example 2 | After | | 1 | 8 | 1 | | 2.1 |
| Example 1 | Before | | 2 | 6 | 1 | 1 | 1.9 |
| | After | 3 | 5 | 2 | | | 0.9 |
| Example 2 | Before | | 1 | 9 | | | 1.9 |
| | After | 3 | 6 | 1 | | | 0.8 |
| Example 3 | Before | | 1 | 7 | 2 | | 2.1 |
| | After | 1 | 9 | | | | 0.9 |

0 = n/a;
1 = has sleep difficulty but barely feels itchy during sleep;
2 = has sleep difficulty and scratches the skin out of awareness during
sleep;
3 = awakened by itch; and
4 = hard to get asleep without sleeping pills.

As can be seen from Table 2, the formulations of Comparative Examples 1 and 2 using either one of the two active components of the present invention alone made no sign of itch relief after their applications. In contrast, the formulations of Examples 1, 2 and 3 using a combination of a high molecular weight poly-gamma-glutamic acid and a low molecular weight poly-gamma-glutamic acid at a content ratio of 1:1, 1:3, and 1:5, respectively, reduced both the intensity and frequency of itch by half.

As a result, the use of a combination of a high molecular weight poly-gamma-glutamic acid and a low molecular weight poly-gamma-glutamic acid at a content ratio of 1:1 to 1:5 according to the present invention created a synergy effect of the two active components to significantly improve the symptoms of atopic dermatitis.

### Experimental Example 2

Skin Patch Stability Test

The formulations of Examples 1, 2 and 3 were evaluated in regards to the stability with skin patches.

A skin patch test was accomplished using Finn Chambers on 33 participants. The back of each participant was cleansed with 70% ethanol and dried out, and 20 μl of the test substance was added dropwise on 8 mm-diameter Finn Chambers. The patches were placed on the back and removed after 24 hours. Skin reactions were read 30 minutes, 24 hours, and 48 hours after application. The procedures were conducted by a dermatologist and the assessment was performed according to the standards of the International Contact Dermatitis Research Group (ICDRG). The assessment results are presented in the following Table 3.

TABLE 3

Skin reaction

| | | | |
|---|---|---|---|
| Div. | Example 1 | Example 2 | Example 3 |

| | | | | |
|---|---|---|---|---|
| 30 | minutes | 0.00 | 0.00 | 0.00 |
| 24 | hours | 0.00 | 0.00 | 0.00 |
| 48 | hours | 0.00 | 0.00 | 0.00 |

As can be seen from Table 3, the composition of the present invention causes a skin reaction of 0.00 in 30 minutes, 24 hours, and 48 hours after the removal of the patches and was considered as “non-stimulant”, causing no skin irritation at all.

### Experimental Example 3

Test for Efficacy of Improving Atopic Skin

The formulation of Example 1 was evaluated in regards to the efficacy of improving atopic skin.

24 male or female participants were selected with an age between 5 to 41 years. All participants were healthy with no acute or chronic disease other than atopic dermatitis. Being selected out of the patients with atopic dermatitis diagnosed according to the Hanifin & Rajka's diagnostic criteria through inquiries by the test supervisor, the participants had a mild-to-severe atopic skin with a scoring atopic dermatitis (SCORAD) index of 15 to 70 and suffered from pruritus (itch) due to dryness. The basic data about the participants are presented in Table 4.

TABLE 4

| Number | Age | Sex |
|---|---|---|
| 1 | 5 | F |
| 2 | 5 | M |
| 3 | 21 | F |
| 4 | 41 | M |
| 5 | 9 | F |
| 6 | 7 | M |
| 7 | 9 | M |
| 8 | 5 | F |
| 9 | 35 | F |
| 10 | 9 | M |
| 11 | 7 | F |
| 12 | 5 | F |
| 13 | 25 | M |
| 14 | 21 | M |
| 15 | 20 | M |
| 16 | 8 | M |
| 17 | 23 | F |
| 18 | 16 | F |
| 19 | 10 | F |
| 20 | 8 | M |
| 21 | 36 | F |
| 22 | 27 | M |
| 23 | 11 | M |
| 24 | 18 | F |
| Average | 15.88 | 12 Males |
| Standard deviation | 10.81 | 12 Females |

The composition of the present invention was applied as a test substance to the atopic skin area designated as a testing area from time to time during the test period of 8 weeks after the face was washed/cleansed or whenever the skin got dry. During the test period, all the participants were forbidden from using a skin moisturizer or any prescribed medication relating to atopic dermatitis such as steroid cream and from receiving operations like pack application or massage.

The measurement of assessment results was conducted after a 30-minute rest in a constant temperature and humidity room (temperature: 22±1° C., humidity: 45±5%) after cleansing the testing area with a same cleanser.

### Experimental Example 3-1

Visual Assessment According to Hanifin & Rajka Criteria

Hanifin & Rajka's diagnostic criteria for atopic dermatitis were employed for a visual assessment on the symptoms of atopic dermatitis, and the scoring atopic dermatitis (SCORAD) index was used as a scale for assessing the severity of atopic dermatitis. The testing area was selected to be available for continuous visual assessment and photography shooting and put into visual assessment. For the SCORAD index, a same person in charge of the testing calculated the SCORAD index according to the calculation criteria by scoring (A) the extent of the affected skin area determined by the area calculation method, (B) the intensity of each symptom, and (C) subjective symptoms in terms of itch and sleeplessness. The area section (A) was scored by evaluating the percentages (%) of the skin areas affected by atopic dermatitis and multiplying the scores of the individual skin areas according to the defined calculation criteria. The intensity section (B) was determined by scoring the severity of six signs (e.g., erythema, oedema/papulation, oozing/crust, excoriation, lichenification, and dryness) on a scale as follows: 0=none; 1=mild; 2=moderate; 3=severe. The subjective symptoms section was determined as the subjective assessment of itch and sleeplessness in the past 3 days on a 10 cm visual analogue scale (VAS) with 0 for no symptom and 10 for most severe symptom.

A digital camera (EOS 540D, Canon, Japan) was used in the photography shooting. For consistent shooting, the positions of the participant and the camera were fixed to maintain a same distance between the testing area and the camera lens, and a same person in charge of the testing measured the skin areas of all the participants affected by atopic dermatitis at a same position with a uniform lighting using a strobe. The reduction of the SCORAD index after the use of the test substance was considered as the improvement of the symptoms of atopic dermatitis. The visual assessment according to the Hanifin & Rajka's diagnostic criteria for atopic dermatitis was performed before treatment with the test substance and at 4 weeks and 8 weeks after treatment.

The calculation formula of the SCORAD index is defined as follows:

SCORAD=A/5+7B/2+C

In the formula, A is the extent of the affected skin determined by the area calculation method; B is the intensity of the symptom; and C is the subjective symptoms in terms of itch and sleeplessness in the past 3 days.

According to an analysis for the improvement of the symptoms of atopic dermatitis on the test skin areas of all participants with atopic dermatitis, the SCORAD index was reduced by 22.06% after 4-week treatment with the test substance and 41.34% after 8-week treatment. The reduction in the SCORAD index was also statistically significant at 4 weeks and 8 weeks after treatment (p<0.001), which revealed that the test substance contributed to the improvement of the symptoms of atopic dermatitis (Refer to Table 5).

TABLE 5
(1) Change of SCORAD index (N = 24)

| | Before | 4 weeks | 8 weeks |
|---|---|---|---|
| Average | 34.17 | 26.63 | 20.04 |
| Standard deviation | 8.46 | 7.52 | 7.55 |

(2) SCORAD index improvement (%)

| | 4 weeks | 8 weeks |
|---|---|---|
| Improvement (%) | 22.06 | 41.34 |

$Improvement ( \% ) = \frac{\vert Measurement after treatment - Measurement before treatment \vert}{Measurement before treatment} \times 100$
(3) Statistical analysis of SCORAD index

| | 4 weeks | 8 weeks |
|---|---|---|
| p-value | .000*** | .000*** |

*p < .05 **p < .01 ***p < .001: p-value is measured by paired t-test.

### Experimental Example 3-2

Assessment on Efficacy of Improving Epidermal Water Content According to Epsilon E100

Epsilon E100 (Biox Systems Ltd., UK) was employed to evaluate the test substance in regards to the function of improving the epidermal water content. The water content of the skin surface in contact with the Epsilon E100 sensor was calculated to an epsilon (E) value. The higher epidermal water content was indicated by the higher brightness of the image so that the blue part got whitish. A same person in charge of the testing evaluated the selected testing areas of all the participants among the areas of the skin affected by atopic dermatitis. For this analysis, the change of the skin water content was analyzed with an analysis program dedicated to Epsilon E100, Epsilon E100 Software Installer V1. The higher measurement value acquired after treatment with the test substance displays that the epidermal water content has improved after the use of the test substance. The instrumental measurement was accomplished before and immediately after treatment with the test substance and at 4 weeks and 8 weeks after treatment.

As a result, the epidermal water content was increased by 215.37% immediately after the treatment with the test substance, 75.80% after 4-week treatment, and 79.47% after 8-week treatment (Refer to Table 6). The increase in the epidermal water content was also statistically significant immediately after treatment with the test substance and at 4 weeks and 8 weeks after treatment (p<0.001), which revealed that the test substance contributed to the improvement of the epidermal water content.

TABLE 6
(1) Change of epidermal water content (N = 24)

| | | Immediately | | |
|---|---|---|---|---|
| | Before | after use | 4 weeks | 8 weeks |
| Average | 3.63 | 11.46 | 6.39 | 6.52 |
| Standard deviation | 1.77 | 4.23 | 2.74 | 2.78 |

ε = Epsilon
(2) Epidermal water content improvement (%)

| | Immediately after use | 4 weeks | 8 weeks |
|---|---|---|---|
| Improvement (%) | 215.37 | 75.80 | 79.47 |

$Improvement ( \% ) = \frac{\vert Measurement after treatment - Measurement before treatment \vert}{Measurement before treatment} \times 100$
(3) Statistical analysis of Epidermal water content

| | Immediately after use | 4 weeks | 8 weeks |
|---|---|---|---|
| p-value | .000*** | .000*** | .000*** |

*p < .05 **p < .01 ***p < .001: p-value is measured by paired t-test.

### Experimental Example 3-3

Assessment on Efficacy of Improving Transepidermal Water Loss with Vapometer

The Vapometer (Delfin, Technologies Ltd., Finland) was employed to evaluate the test substance in regards to the function of improving the transepidermal water loss. A same person in charge of the testing put the probe in contact with the selected testing areas of all the participants among the areas of the skin affected by atopic dermatitis under the same pressure to measure the transepidermal water loss. The Vapometer has a humidity sensor that is inside a measurement chamber to measure the evaporation rate from the increase of relative humidity (RH) inside the chamber during the measurement. The measurement unit is g/m²/h. The lower measurement value acquired after the treatment with the test substance displays that the transepidermal water loss has improved after the use of the test substance. The instrumental measurement was accomplished before and immediately after treatment with the test substance and at 4 weeks and 8 weeks after treatment.

As a result, the transepidermal water loss was decreased by 15.76% immediately after the treatment with the test substance, 30.90% after 4-week treatment, and 41.33% after 8-week treatment. The decrease in the transepidermal water loss was also statistically significant immediately after the treatment with the test substance and at 4 weeks and 8 weeks after treatment (p<0.01), which revealed that the test substance contributed to the improvement of the transepidermal water loss (Refer to Table 7).

TABLE 7
(1) Change of transepidermal water loss (N = 24)

| | | Immediately | | |
|---|---|---|---|---|
| | Before | after use | 4 weeks | 8 weeks |
| Average | 28.44 | 23.96 | 19.65 | 16.69 |
| Standard deviation | 20.29 | 18.51 | 15.20 | 9.22 |

g/m²/h
(2) Transepidermal water loss improvement (%)

| | Immediately after use | 4 weeks | 8 weeks |
|---|---|---|---|
| Improvement (%) | 15.76 | 30.90 | 41.33 |

$Improvement ( \% ) = \frac{\vert Measurement after treatment - Measurement before treatment \vert}{Measurement before treatment} \times 100$
(3) Statistical analysis of Transepidermal water loss

| | Immediately after use | 4 weeks | 8 weeks |
|---|---|---|---|
| p-value | .000*** | .002*** | .004*** |

*p < .05 **p < .01 ***p < .001: p-value is measured by paired t-test.

### Experimental Example 3-4

Assessment on Efficacy of Improving Desquamation (Scaling Skin) Using Video Microscope and Image Analysis Program

A video microscope (Skin Diagnosis System SDM, Bomtech, Korea) and an image analysis program (Image J, National Institutes of Health, USA) were used to evaluate the test substance in regards to the function of improving desquamation (scaling skin). For consistent shooting, a same person in charge of the testing operated the video microscope to take images (magnified to 250× their original size) of the selected skin areas of all the participants affected by atopic dermatitis at a same position with a uniform lighting. The images thus obtained were processed through image-thresholding and reduced to a binary image according to the Otsu's method. In the binary image, the white portion above the threshold value was considered as skin scales (dead skin cells), and its proportion to the total skin area was calculated. The lower measurement value acquired after the treatment with the test substance displays that the intensity of desquamation (scaling skin) has improved through the use of the test substance. The instrumental measurement was accomplished before and immediately after treatment with the test substance and at 4 weeks and 8 weeks after treatment.

As a result, the intensity of desquamation (scaling skin) was reduced by 62.87% immediately after the treatment with the test substance, 55.84% after 4-week treatment, and 63.47% after 8-week treatment. The reduction of desquamation was also statistically significant immediately after treatment with the test substance and at 4 weeks and 8 weeks after treatment (p<0.001), which revealed that the test substance contributed to the improvement of desquamation (Refer to Table 8).

TABLE 8
(1) Change in severity of desquamation (scaling skin) (N = 24)

| | | Immediately | | |
|---|---|---|---|---|
| | Before | after use | 4 weeks | 8 weeks |
| Average | 26.07 | 9.68 | 11.51 | 9.52 |
| Standard deviation | 9.30 | 3.66 | 5.84 | 5.84 |

%
(2) Desquamation improvement (%)

| | Immediately after use | 4 weeks | 8 weeks |
|---|---|---|---|
| Improvement (%) | 62.87 | 55.84 | 63.47 |

$Improvement ( \% ) = \frac{\vert Measurement after treatment - Measurement before treatment \vert}{Measurement before treatment} \times 100$
(3) Statistical analysis on severity of desqamation

| | Immediately after use | 4 weeks | 8 weeks |
|---|---|---|---|
| p-value | .000*** | .000*** | .000*** |

*p < .05 **p < .01 ***p < .001: p-value is measured by paired t-test.

### Experimental Example 3-5

Assessment on Skin Soothing Effect Using Mexameter

Mexameter (Mexameter MX18, Courage-Khazaka Electronic GmbH, Cologne, Germany) was used to evaluate the test substance in regards to the skin soothing effect. A same person in charge of the testing conducted the measurement three consecutive times on a same point of the selected skin areas of all the participants affected by atopic dermatitis under a same pressure. The three measurements were averaged and the average value was applied to the analysis. The Mexameter emits radiations of different wavelength ranges corresponding to melanin and hemoglobin (erythema) and reduces the intensity of the reflected radiations to values according to the optical measurement technique. The measurement value consists of a melanin index and an erythema index. The melanin index indicates the quantity of melanin present in the skin; and the erythema index indicates the quantity of hemoglobin in the skin. The erythema index describing the intensity of skin redness was used as a measurement value indicating the skin soothing effect of the test substance. The lower measurement value acquired after the treatment with the test substance displays that the test substance makes a skin soothing effect. The instrumental measurement was accomplished before and immediately after treatment with the test substance and at 4 weeks and 8 weeks after treatment.

As a result, the erythema index indicating the intensity of skin redness was reduced by 14.60% after 4-week treatment with the test substance and 19.68% after 8-week treatment. The reduction of erythema index was also statistically significant at 4 weeks and 8 weeks after treatment (p<0.001), which revealed that the test substance contributed to the skin soothing effect (Refer to Table 9).

TABLE 9
(1) Change of erythema index (N = 24)

| | Before | 4 weeks | 8 weeks |
|---|---|---|---|
| Average | 278.48 | 237.83 | 223.68 |
| Standard deviation | 66.64 | 64.45 | 54.97 |

(2) ΔErythema index improvement (%)

| | ΔErythema index₁ | ΔErythema index₂ |
|---|---|---|
| Average | −40.65 | −54.80 |
| Standard deviation | 38.78 | 43.28 |

ΔErythema index₁ = Erythema index at 4 weeks − Erythema index before treatment
ΔErythema index₂ = Erythema index at 8 weeks − Erythema index before treatment
(3) Erythema index improvement (%)

| | 4 weeks | 8 weeks |
|---|---|---|
| Improvement (%) | 14.60 | 19.68 |

$Improvement ( \% ) = \frac{\vert Measurement after treatment - Measurement before treatment \vert}{Measurement before treatment} \times 100$
(4) Statistical analysis of erythema index

| | 4 weeks | 8 weeks |
|---|---|---|
| p-value | .000*** | .000*** |

*p < .05 **p < .01 ***p < .001: p-value is measured by paired t-test.

### Experimental Example 3-6

Assessment on Efficacy of Relieving Pruritus (Itch) Caused by Dryness

(1) Assessment on the Efficacy of Improving Dermal Moisturization Using MoistureMeterD Compact

MoistureMeterD Compact (Delfin Technologies Ltd., Finland) was used to evaluate the test substance in regards to the function of improving dermal moisturization among the assessments on the improvement of pruritus (itch) caused by dryness. In the assessment procedures, a same person in charge of the testing put the probe vertically positioned in contact with the selected skin areas of all the participants affected by atopic dermatitis and measured the dermal water content while maintaining the horizontal level for 10 seconds. The MoistureMeterD Compact measures the tissue dielectric constant (TDC) in the skin with a measurement depth of 2 mm at a wavelength of 300 MHz. The TDC measurement unit is percentage (%). The higher measurement value acquired after the treatment with the test substance displays that dermal moisturization has improved through the use of the test substance. The instrumental measurement was accomplished before and immediately after treatment with the test substance and at 4 weeks and 8 weeks after treatment.

As a result, the dermal water content was increased by 21.87% immediately after treatment with the test substance, 11.66% after 4-week treatment, and 18.47% after 8-week treatment. The increase in the dermal water content was also statistically significant immediately after treatment with the test substance and at 4 weeks and 8 weeks after treatment (p<0.01), which revealed that the test substance contributed to the improvement of dermal moisturization (Refer to Table 10).

TABLE 10
(1) Change in dermal water content (N = 24)

| | | Immediately | | |
|---|---|---|---|---|
| | Before | after use | 4 weeks | 8 weeks |
| Average | 34.29 | 41.79 | 38.29 | 40.63 |
| Standard deviation | 7.45 | 8.30 | 8.17 | 8.52 |

%
(2) Dermal water content improvement (%)

| | Immediately after use | 4 weeks | 8 weeks |
|---|---|---|---|
| Improvement (%) | 21.87 | 11.66 | 18.47 |

$Improvement ( \% ) = \frac{\vert Measurement after treatment - Measurement before treatment \vert}{Measurement before treatment} \times 100$
(3) Statistical analysis on dermal water content

| | Immediately after use | 4 weeks | 8 weeks |
|---|---|---|---|
| p-value | .000*** | .004** | .004*** |

*p < .05 **p < .01 ***p < .001: p-value is measured by paired t-test.

(2) Questionnaire-Based Assessment of the Function of Relieving Pruritus (Itch) Caused by Dryness

This experiment used 5-item questionnaires to evaluate the behavior and habit in association with pruritus before and after treatment with the test substance, and 3-item questionnaires to evaluate the mental state in association with pruritus before and after treatment with the test substance on a 10 cm visual analogue scale (VAS) with 0 for no symptom and 10 for most severe symptom.

The statistical analysis in the present example was accomplished with an SPSS 17.0 for Windows program. A Wilcoxon-signed ranks analysis was performed to analyze questionnaire survey on the relief of pruritus (itch) caused by dryness. The general analysis of the questionnaire survey used statistics including average, standard deviation, frequency, and percentage. In addition, the paired t-test was adopted to determine whether there is a significant difference between different instrumental measurements concerning skin improvement.

As a result, as shown in Table 11, the intensity of pruritus (itch) reduced from 3.25 before treatment with the test substance to 2.17 after 4-week treatment and 1.25 after 8-week treatment; the frequency of pruritus (itch) decreased from 2.88 before treatment with the test substance to 1.58 after 4-week treatment and 1.00 after 8-week treatment; the intensity of pruritus (itch) during sleep reduced from 2.54 before treatment with the test substance to 1.33 after 4-week treatment and 0.67 after 8-week treatment; the intensity of the worst imaginable imaginable pruritus (itch) reduced from 3.88 before treatment with the test substance to 2.54 after 4-week treatment and 1.50 after 8-week treatment; and the intensity of the mildest imaginable pruritus (itch) reduced from 2.58 before treatment with the test substance to 1.29 after 4-week treatment and 0.79 after 8-week treatment. The reduction of pruritus (itch) intensity was also statistically significant immediately at 4 weeks and 8 weeks after treatment with the test substance (p<0.001), which revealed that the test substance contributed to the improvement of behavior and habit in association with pruritus.

TABLE 11
Change of behavior and habit in association with pruritus (itch)

(N = 24)

(1) Questions for pruritus (itch) intensity

| Ques- | Before | | 4 Weeks | | 8 Weeks | |
|---|---|---|---|---|---|---|
| tion | Frequency | Average | Frequency | Average | Frequency | Average |
| 0 | 0 | 3.25 | 0 | 2.17 | 2 | 1.25 |
| 1 | 0 | | 2 | | 15 | |
| 2 | 2 | | 17 | | 6 | |
| 3 | 14 | | 4 | | 1 | |
| 4 | 8 | | 1 | | 0 | |
| 5 | 0 | | 0 | | 0 | |

0 = n/a;
1 = nearly feels neither itchy nor unpleasant;
2 = sometimes feels slightly itchy;
3 = frequently feels itchy but pleasant for activity or rest;
4 = frequently feels extreme itch that is disturbing for relaxation but not
for activity; and
5 = always feels extreme itch that disturbs both activity and relaxation

(N = 24)

(2) Questions for pruritus (itch) frequency

| Ques- | Before | | 4 Weeks | | 8 Weeks | |
|---|---|---|---|---|---|---|
| tion | Frequency | Average | Frequency | Average | Frequency | Average |
| 0 | 0 | 2.88 | 0 | 1.58 | 3 | 1.00 |
| 1 | 1 | | 13 | | 18 | |
| 2 | 8 | | 8 | | 3 | |
| 3 | 10 | | 3 | | 0 | |
| 4 | 3 | | 0 | | 0 | |
| 5 | 2 | | 0 | | 0 | |

0 = n/a;
1 = 3 or less times a day, lasting for 10 minutes or less on each time;
2 = 4 or more times a day, lasting for 10 minutes or less on each time;
3 = 4 or more times a day, lasting for 15 minutes or longer on each time;
4 = 4 or more times a day, lasting for 30 minutes or less on each time; and
5 = incessant itch

(N = 24)

(3) Questions for pruritus (itch) intensity during sleep

| Ques- | Before | | 4 Weeks | | 8 Weeks | |
|---|---|---|---|---|---|---|
| tion | Frequency | Average | Frequency | Average | Frequency | Average |
| 0 | 0 | 2.54 | 3 | 1.33 | 11 | 0.67 |
| 1 | 1 | | 11 | | 11 | |
| 2 | 9 | | 9 | | 1 | |
| 3 | 14 | | 1 | | 1 | |
| 4 | 0 | | 0 | | 0 | |

0 = n/a;
1 = has sleep difficulty but barely feels itchy during sleep;
2 = has sleep difficulty and scratches the skin out of awareness during
sleep;
3 = awakened by itch; and
4 = hard to get asleep without sleeping pills

(N = 24)

(4) Questions for intensity of worst imaginable pruritus (itch)

| Ques- | Before | | 4 Weeks | | 8 Weeks | |
|---|---|---|---|---|---|---|
| tion | Frequency | Average | Frequency | Average | Frequency | Average |
| 0 | 0 | 3.88 | 0 | 2.54 | 1 | 1.50 |
| 1 | 0 | | 2 | | 12 | |
| 2 | 1 | | 12 | | 10 | |
| 3 | 4 | | 5 | | 0 | |
| 4 | 16 | | 5 | | 1 | |
| 5 | 3 | | 0 | | 0 | |

0 = n/a;
1 = often feels itchy, but does not need to scratch
2 = often scratches without damaging skin
3 = continuously scratches without damaging skin
4 = needs to scratch so hard to nearly damage skin
5 = needs to scratch continuously even on damaged skin

(N = 24)

(5) Questions for intensity of mildest imaginable pruritus (itch)

| Ques- | Before | | 4 Weeks | | 8 Weeks | |
|---|---|---|---|---|---|---|
| tion | Frequency | Average | Frequency | Average | Frequency | Average |
| 0 | 0 | 2.58 | 2 | 1.29 | 7 | 0.79 |
| 1 | 1 | | 14 | | 16 | |
| 2 | 11 | | 7 | | 2 | |
| 3 | 9 | | 1 | | 0 | |
| 4 | 3 | | 0 | | 0 | |
| 5 | 0 | | 0 | | 0 | |

0 = n/a;
1 = often feels itchy, but does not need to scratch
2 = often scratches without damaging skin
3 = continuously scratches without damaging skin
4 = needs to scratch so hard to nearly damage skin
5 = needs to scratch continuously even on damaged skin

### INDUSTRIAL AVAILABILITY

The composition for improving symptoms of atopic dermatitis according to the present invention, when applied on the skin, can inhibit epidermal and transepidermal water loss, improve desquamation (scaling skin), provide an excellent skin soothing effect, and relieve pruritus (itch) caused by dryness, thereby making remarkable improvement of general symptoms of atopic dermatitis.

While the present invention has been particularly illustrated and described with reference to exemplary embodiments thereof, it is apparent to those skilled in the art that the detailed description is of the best currently contemplated modes of carrying out exemplary embodiments of the invention and that the scope of the present invention is not limited to the disclosed embodiment. Accordingly, the substantial scope of the present invention should be defined by the following claims and equivalents thereof.

What is claimed is:

1. A composition for topical use for improving symptoms of atopic dermatitis, the composition comprising as active components:
a high molecular weight poly-gamma-glutamic acid; and
a low molecular weight poly-gamma-glutamic acid.

2. The composition for topical use for improving symptoms of atopic dermatitis as claimed in claim 1, wherein the high molecular weight poly-gamma-glutamic acid and the low molecular weight poly-gamma-glutamic acid are contained at a ratio of 1:1 to 1:5.

3. The composition for topical use for improving symptoms of atopic dermatitis as claimed in claim 1, wherein the high molecular weight poly-gamma-glutamic acid has a molecular weight of 1,500 to 3,500 kDa.

4. The composition for topical use for improving symptoms of atopic dermatitis as claimed in claim 1, wherein the low molecular weight poly-gamma-glutamic acid has a molecular weight of 0.5 to 2 kDa.
